(19) United States
(12) Patent Application Publication
(10) Pub. No.: US 2023/0233339 A1
(43) Pub. Date: Jul. 27, 2023

(54) CONFIGURABLE STIFFNESS SPRING FOR PROSTHESES AND ORTHOSES

(71) Applicant: Little Room Innovations, Ann Arbor, MI (US)

(72) Inventors: Harrison Logan Bartlett, Atlanta, GA (US); Brian Edward Lawson, Ann Arbor, MI (US); Maxwell Keith Shepherd, Charlottesville, VA (US)

(21) Appl. No.: 18/086,227

(22) Filed: Dec. 21, 2022

Related U.S. Application Data
(60) Provisional application No. 63/292,909, filed on Dec. 22, 2021.

(51) Int. Cl. A61F 2/50

(52) U.S. Cl. CPC A61F 2/50; A61F 2/6607

(57) ABSTRACT

Various implementations include a compliant element comprising a spring component, a mounting portion, and a transverse shear constraint element. The spring component comprises two or more stacked beam elements. Each beam element has a first and second end with the first end of each beam is attached to the mounting portion. Furthermore, the transverse shear constraint element is located between the first and second ends of the beam elements and prevents the relative sliding of two or more beam elements in the region of the beams between the first beam ends and the transverse shear constraint element.

### BACKGROUND

The vast majority of prosthetic feet currently available to patients are passive devices, which behave largely as springs, mimicking the spring-like nature of the biological ankle-foot complex during walking. Perhaps the two most dominant variables governing prosthetic foot mechanics are sagittal plane angular alignment and stiffness. Both strongly affect “roll-over,” a term that prosthetists commonly use to describe the support and smoothness of tibial progression through midstance, particularly as an analogy of a “rocker foot.” However, there are key differences between these variables, in terms of energy storage and return, foot clearance with the ground, and neutral angle during standing.

The current clinical fitting process involves substantial time and effort optimizing alignment, but effectively zero time on optimizing stiffness. This is partially due to the way prosthetic feet are ordered—once a foot arrives, it can be aligned, but stiffness cannot be changed. It is uncommon in the United States for prosthetists to have multiple stiffness levels on hand for subjects to try, largely due to the way reimbursement for prosthetic feet works. Rather, they order the foot for a particular patient, relying heavily on manufacturer recommendations, which are based solely on patient weight and activity level.

Recent work has shown that, when appropriately exposed to variations in prosthesis mechanics and trained on providing feedback, below-knee amputees provide consistent preferences for both stiffness and alignment. The exposure to these variations (i.e., exploring the landscape of mechanics) is a requirement for developing and communicating preferences. Unfortunately, prosthetists are unable to modify certain variables, such as stiffness, meaning they cannot train subjects to provide feedback on those variables.

Other industries for assistive technologies involve patient-centric tuning of the device, with perhaps the most notable example being in the field of optometry/ophthalmology (specifically in the prescription of eyeglasses). During the process of eyeglass prescription, a clinical tool known as the phoropter is used to allow patients to explore the landscape of lens refractive power. In these fields, patients are relied upon to provide quick and honest feedback on their preferences, often in an A-B style comparison (or, two alternative forced choice task). Responses provide one bit of information, allowing a constant size step in the correct direction. These approaches are often informally used in adjusting prosthesis alignment, where prosthetists pose questions about what the patient prefers. In these and similar scenarios, the ability to immediately adjust the mechanics is a requirement.

A major reason for the nonexistence of a clinical instrument for prosthetic foot prescription that parallels the phoropter—the instrument used by optometrists—is that it is intrinsically difficult to design a robust, lightweight, low-profile prosthetic foot capable of quickly varying stiffness over a wide range. Prosthetic feet are almost solely made from modern composite materials—namely carbon fiber and fiberglass—which are the only known materials capable of storing the requisite elastic energy in a light and anthropometric shape. A few research devices have been able to change stiffness by way of replacing springs within the foot, but the process of replacing springs takes too long for patients to perform precise A-B comparisons, the keel and heel are often not independently adjustable, and they do not fit in an anthropometric foot shell and cannot be worn with shoes.

Several current prosthetic and orthotic devices with variable stiffness focus on varying the rotational stiffness of a prosthetic or orthotic joint about a single fixed axis of rotation. However, most commercially available compliant prosthetic feet do not have a single axis of rotation. Instead, most commercially available prosthetic feet are constructed as cantilevered beam-like structures, which, when subject to loads, undergo a distributed bending deformation. As such, current commercially available prosthetic devices do not have a single axis of rotation and cannot be accurately emulated by devices with a single axis of rotation. Furthermore, most prosthetic and orthotic devices described in prior art vary their stiffness through the use of a propped cantilevered beam mechanism in which a cantilevered beam serves as the compliant element, and a support point is placed between the beam and a stationary, grounded structure at a point between the fixed and free ends of the beam. This propped cantilevered beam mechanism requires that a stationary, grounded structure is present along the length of the cantilevered beam, to prevent the motion of the support point. This grounded structure is typically large in order to withstand the loads placed on the support point. As such, a propped cantilevered beam mechanism requires substantial material, size, and weight to achieve stiffness variation. Such deficiencies limit their ability to emulate commercially available prosthetic feet while maintaining a compact and lightweight package.

Thus, a need exists for a clinical instrument for prosthetic foot prescription including a prosthetic device with adjustable physical parameters.

### SUMMARY

Various implementations include a compliant element for an ankle and foot prosthesis or orthosis. The compliant element includes a mounting portion, a spring component, and a transverse shear constraint element. The spring component includes two or more stacked beam elements. Each beam element has a first end and a second end opposite and spaced apart from the first end. The first end of each of the beam elements is fixedly attached to the mounting portion. The transverse shear constraint element is located between the first end and the second end of the beam elements. The transverse shear constraint element prevents relative sliding of at least two beam elements in a region of the beam elements between the first end of the beam elements and the transverse shear constraint element.

In some implementations, the transverse shear constraint element is movable between the first end and the second end of the beam elements.

In some implementations, the transverse shear constraint element prevents the relative sliding of all of the two or more stacked beam elements in the region of the beam elements between the first end of the beam elements and the transverse shear constraint element.

In some implementations, the transverse shear constraint element prevents the relative sliding of a subset of the beam elements between the first end and the second end of the beam elements. In some implementations, the transverse shear constraint element is configured to selectively engage various subsets of beam elements.

In some implementations, the transverse shear constraint element includes a clamping mechanism. In some implementations, the transverse shear constraint element is a clamp that is actuated by a cam mechanism.

In some implementations, at least one of the beam elements includes a different material from at least one other beam element.

In some implementations, the beam elements have a constant thickness between the first end and the second end of the beam elements. In some implementations, the beam elements are straight. In some implementations, the beam elements are curvilinear.

Various implementations include a prosthetic foot including a compliant element such as the compliant elements described above.

In some implementations, the prosthetic foot includes a hindfoot component and a forefoot component. In some implementations, the compliant element includes at least one of the hindfoot component or the forefoot component.

Various implementations include an ankle foot orthosis including a compliant element such as the compliant elements described above. In some implementations, the ankle foot orthosis includes a shank component and a foot component. In some implementations, the compliant element is coupled to one of the shank component or the foot component at the mounting portion and to the other of the foot component or the shank component at the second end of at least one of the beam elements.

### BRIEF DESCRIPTION OF DRAWINGS

Example features and implementations are disclosed in the accompanying drawings. However, the present disclosure is not limited to the precise arrangements and instrumentalities shown.

FIGS. 1A and 1B are side views of a compliant element having stiffness variation, according to one implementation. The spring element is stiff when the transverse shear constraint element is located near the second ends of the beam elements (A) and is compliant when the transverse shear constraint element is located near the first end of the beam elements (B).

FIG. 2 is a perspective view of a prosthetic foot including the compliant element of FIG. 1 with adjustable stiffness forefoot.

FIG. 3 is a sagittal side view of the prosthetic foot of FIG. 2 in a compliant configuration.

FIG. 4 is a sagittal side view of the prosthetic foot of FIG. 2 in a stiff configuration.

FIG. 5 is a sagittal side view of a prosthetic foot including a compliant element, according to another implementation, including a clamp-based transverse shear constraint element driven by a cam mechanism.

FIG. 6 is a sagittal side view of a prosthetic foot including a compliant element, according to another implementation, including a clamp mechanism that is actuated by a screw which is driven along an axis that is different than that of the clamping direction. The screw is driven perpendicular to the clamping direction and the clamp is driven by a system of wedges.

FIG. 7 is a sagittal side view of a prosthetic foot including a compliant element, according to another implementation, including a shear pin inserted into the faces of adjacent beams to serve as transverse shear constraint element.

FIG. 8 is a perspective view of a prosthetic foot including a compliant element, according to another implementation, including shear pins inserted into the sides of adjacent beams to serve as transverse shear constraint element.

FIG. 9 is a perspective view of a two material beam element, according to various implementations.

FIG. 10 is a side view of a tall build height prosthetic foot, according to another implementation.

FIG. 11 is a side view of a posterior mounted prosthetic foot, according to another implementation.

FIG. 12 is a side view of an ankle foot orthosis with adjustable stiffness, according to another implementation.

### DETAILED DESCRIPTION

The devices, systems, and methods disclosed herein provide for a mechanism that allows for the adjustment of the bending stiffness of a compliant element while maintaining a simple and compact form. The devices, systems, and methods disclosed herein allow for the bending stiffness of a compliant element to be varied, and as such, may have applications in a variety of both prosthetic and orthotic applications. This compliant element with configurable stiffness may be used in a forefoot, hindfoot, or both to address this clinical need. The devices, systems, and methods disclosed herein utilize a novel method of varying stiffness that allows a prosthetic or orthotic device to experience a distributed bending deformation (rather than a rotation about a single axis of rotation) while maintaining a small form factor (minimizing both size and weight).

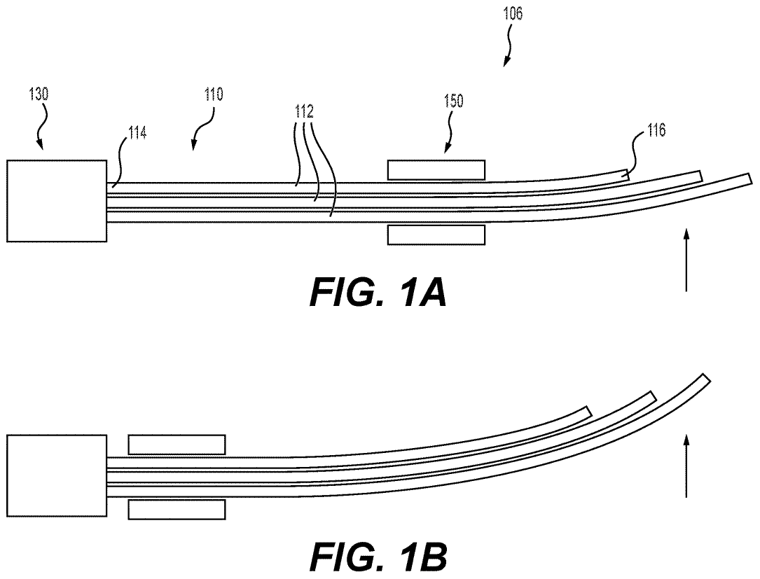

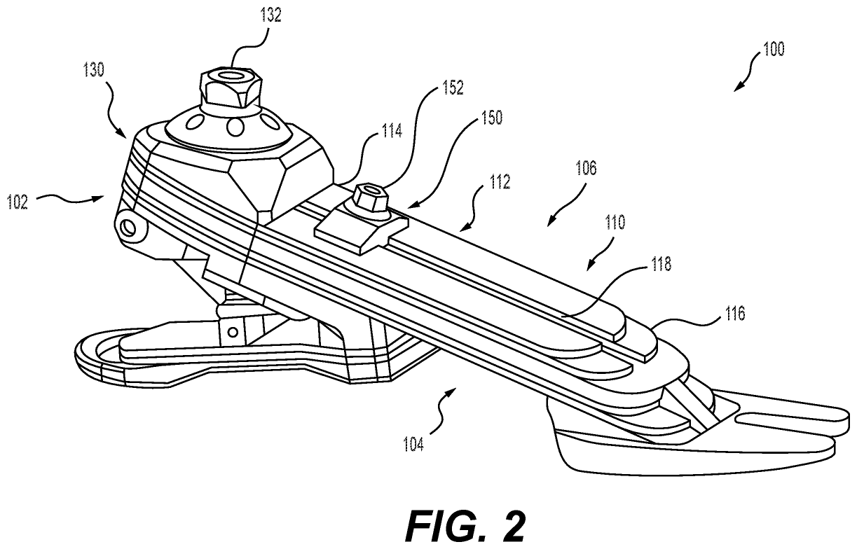

FIGS. 1-4 show a prosthetic foot 100 including a hindfoot component 102 and a forefoot component 104. The hindfoot component 102 includes a heel. The heel can be adjustable, as shown in FIGS. 2-4, or static.

The forefoot component 104 shown in FIGS. 1-4 includes a compliant element 106. The compliant element 106 includes a mounting portion 130, a spring component 110, and a transverse shear constraint element 150. The mounting portion 130 shown in FIGS. 1-4 includes a shank component or shank body segment 132. However, in some implementations, the mounting portion includes a prosthetic socket.

The spring component 110 includes two or more stacked beam elements 112. Each beam element 112 has a first end 114 and a second end 116 opposite and spaced apart from the first end 114. The first end 114 of each beam element 112 is fixedly coupled to the mounting portion 130 such that the first ends 114 of each beam element 112 remain static relative to the first ends 114 of the other beam elements 112. Each of the beam elements 112 defines a slot 118 that, when the beam elements 112 are stacked, the slots 118 of each of the beam elements 112 are aligned.

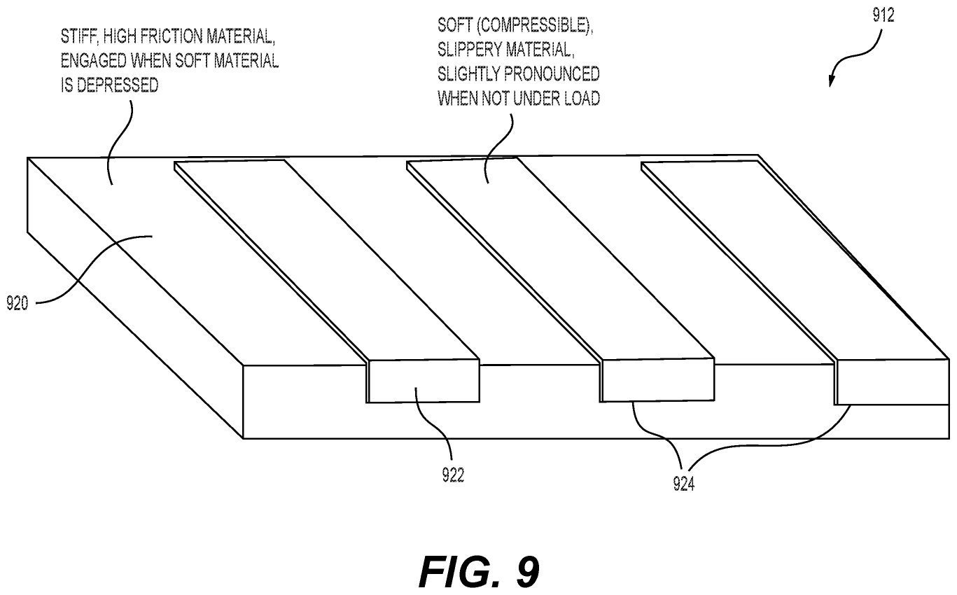

The beam elements 112 shown in FIGS. 1-4 are made of a single material. However, as shown in FIG. 9, the beam elements 912 are made of a first material 920 and a second material 922. The first material 920 of the beam elements 912 in FIG. 9 includes a relatively higher friction material. The beam elements 912 define openings 924 in which relatively the soft and compressible second material 922 is embedded. When a clamping force is applied to these beam elements 912, the soft second material 922 is compressed, and the adjacent high friction first material 920 makes contact. In some implementations, the beam elements include three or more different materials.

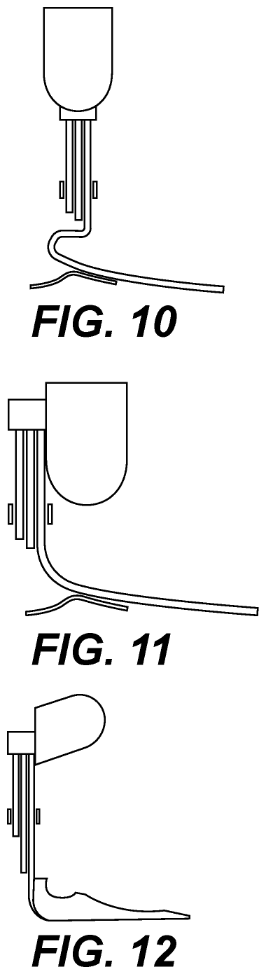

The beam elements 112 shown in FIGS. 1-4 are straight and have a constant thickness from the first end 114 to the second end 116. However, in some implementations, the beam elements have variable thickness. In some implementations, such as those shown in FIGS. 10-12, the beam elements are curvilinear.

The transverse shear constraint element 150 includes a screw 152 that extends through the aligned slots 118 of the beam elements 112. The transverse shear constraint element 150 is located between the first end 114 and the second end 116 of the beam elements 112. When the screw 152 is tightened, portions of the transverse shear constraint element 150 compress the stack of beam elements 112 to prevent the relative sliding of all of the beam elements 112 in the region of the beam elements 112 between the first end 114 of the beam element 112 and the transverse shear constraint element 150. The region of the beam elements 112 between the first end 114 of the beam element 112 and the transverse shear constraint element 150 in which the beam elements 112 are prevented from sliding relative to each other has a relatively high stiffness compared to the regions of the individual beam elements 112 between the transverse shear constraint element 150 and the second ends 116 of the beam elements 112 in which the beam elements 112 are able to slide relative to each other and, therefore, bend individually.

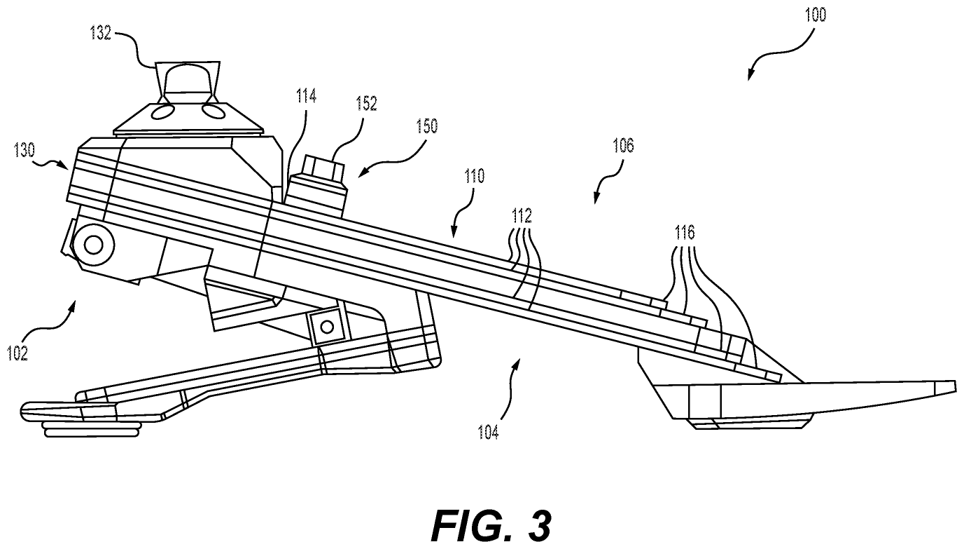

The transverse shear constraint element 150 shown in FIGS. 1-4 is movable between the first end 114 and the second end 116 of the beam elements 112 to adjust the stiffness of the compliant element 100. As the transverse shear constraint element 150 is moved closer to the first end 114 of the beam elements 112, as shown in FIG. 3, the lengths of the beam elements 112 between the transverse shear constraint element 150 and the second ends 116 of the beam elements 112 that are able to slide relative to each other become longer. The longer individual beam elements 112 allow for more leverage in use and, thus, are easier to bend. In effect, this makes the compliant element 106 have a relatively higher flexibility.

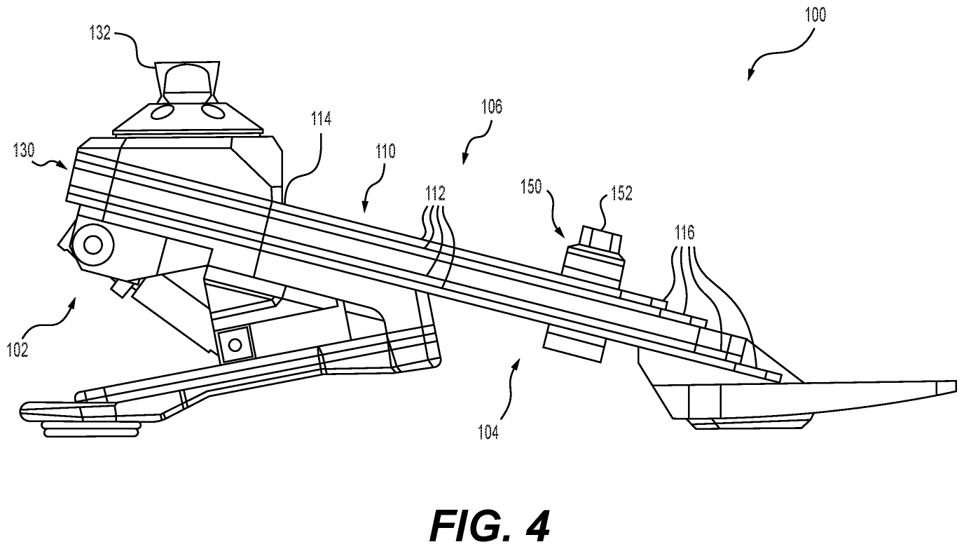

When the transverse shear constraint element 150 is moved closer to the second end 116 of the beam elements 112, as shown in FIG. 4, the lengths of the beam elements 112 between the transverse shear constraint element 150 and the second ends 116 of the beam elements 112 that are able to slide relative to each other become shorter. The shorter individual beam elements 112 allow for less leverage in use and, thus, are harder to bend. In effect, this makes the compliant element 106 have a relatively lower flexibility.

Although the transverse shear constraint element 150 shown in FIGS. 1-4 prevents the relative sliding of all of the stacked beam elements 112 in the region of the beam elements 112 between the first end 114 of the beam elements 112 and the transverse shear constraint element 150, in some implementations, the transverse shear constraint element prevents the relative sliding of only two or more of the stacked beam elements in the region of the beam elements between the first end of the beam elements and the transverse shear constraint element.

In some implementations, the transverse shear constraint element prevents the relative sliding of a subset of the beam elements between the first end and the second end of the beam elements. In some implementations, the transverse shear constraint element is configured to selectively engage various subsets of beam elements.

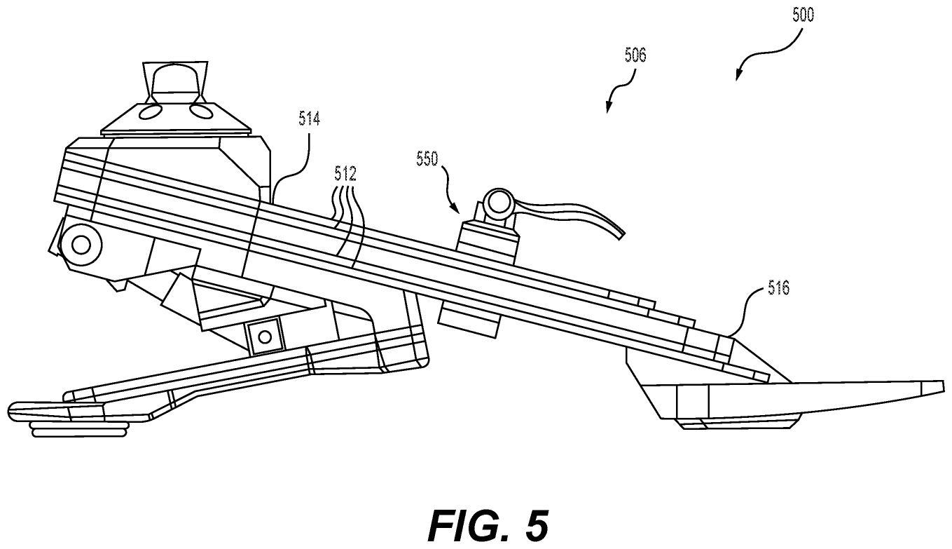

In some implementations, the transverse shear constraint element includes any other mechanism for preventing the relative sliding of regions of two or more beam elements between the first ends of the beam elements and the transverse shear constraint element. For example, the prosthetic foot 500 shown in FIG. 5 includes a transverse shear constraint element 550 according to another implementation. The transverse shear constraint element 550 in the compliant element 506 shown in FIG. 5 is a clamp that is actuated by a cam mechanism. When the cam mechanism is in the open position, the transverse shear constraint element 550 is slidable between the first end 514 and the second end 516 of the beam elements 512. In the closed position, the cam mechanism of the clamp compresses the beam elements 512 to prevent the relative sliding of all of the beam elements 512 in the region of the beam elements 512 between the first end 514 of the beam elements 512 and the transverse shear constraint element 550. Although the clamp shown in FIG. 5 is a cam mechanism, in some implementations, the transverse shear constraint element includes any type of clamping mechanism.

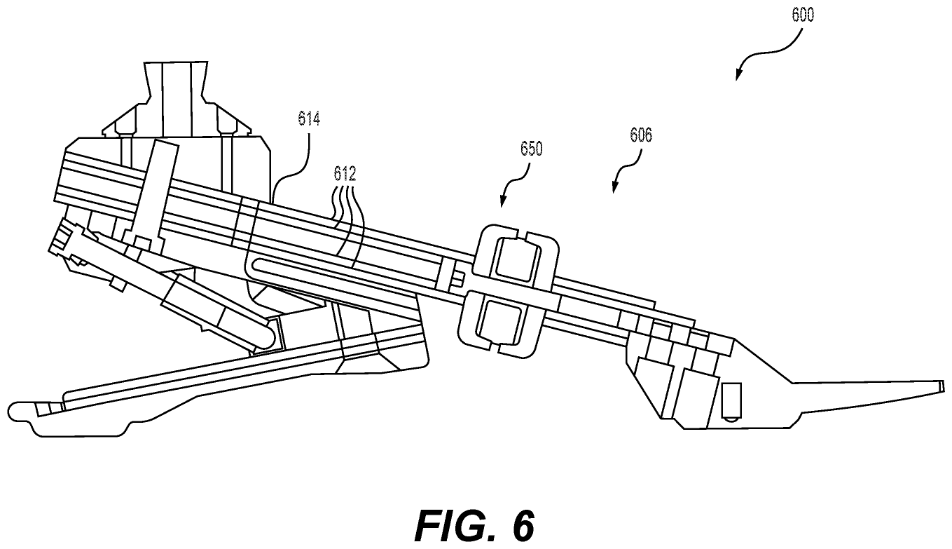

The prosthetic foot 600 shown in FIG. 6 includes another implementation of a transverse shear constraint element 650. The transverse shear constraint element 650 in the compliant element 606 shown in FIG. 6 includes a screw that extends parallel to the surfaces of the beam elements 612. Tightening of the screw of the transverse shear constraint element 650 causes the compression of the stacked beam elements 612 to prevent the relative sliding of all of the beam elements 612 in the region of the beam elements 612 between the first end 614 of the beam elements 612 and the transverse shear constraint element 650.

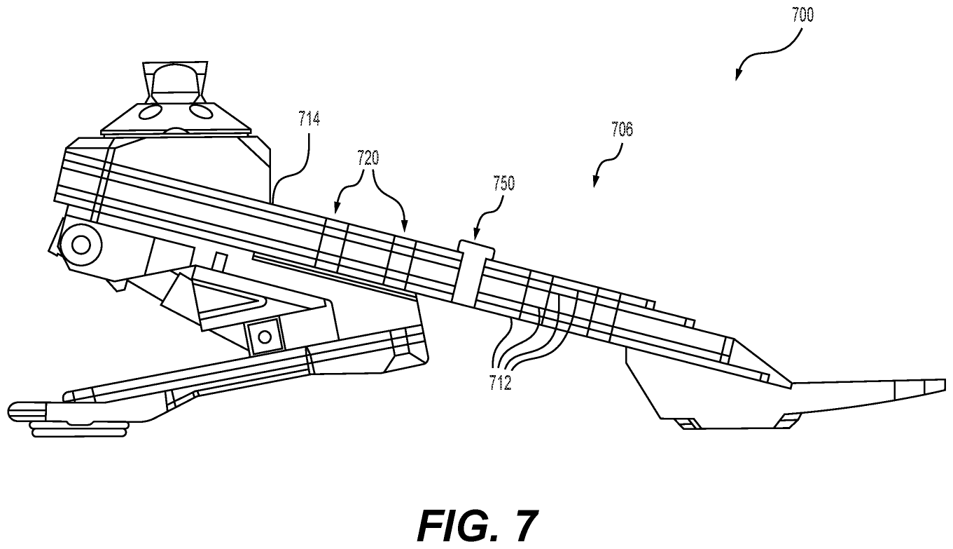

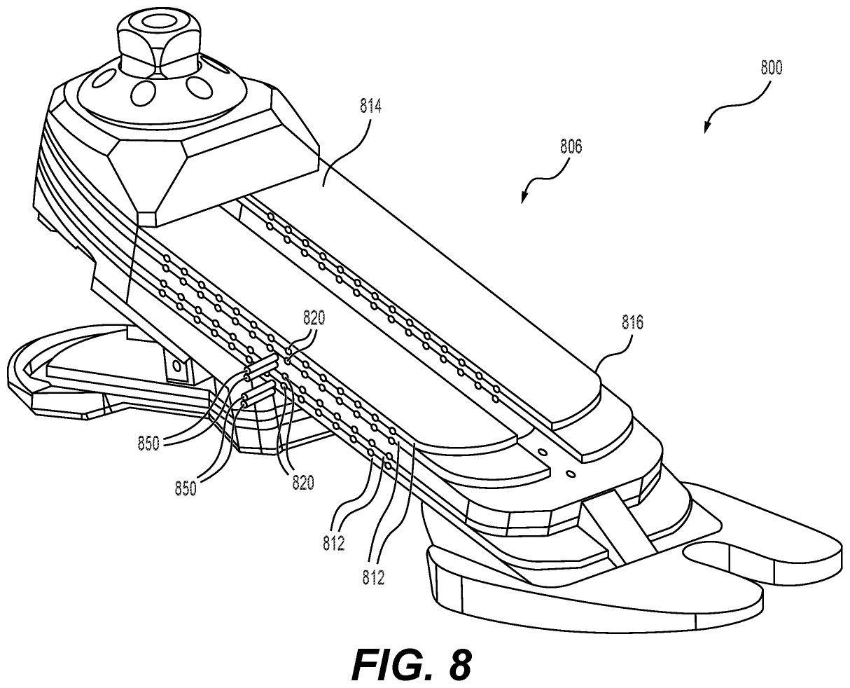

The prosthetic feet 700, 800 shown in FIGS. 7 and 8 include two other implementations of transverse shear constraint elements 750, 850. The beam elements 712 shown in FIG. 7 each include openings 720 extending perpendicular to the surfaces of each of the beam elements 712, wherein each of the openings 720 in a beam element 712 are axially alignable with the openings 720 in the other beam elements 712. The transverse shear constraint element 750 in the compliant element 706 shown in FIG. 7 is a pin that is disposable within a set of alignable openings 720 of the stack of beam elements 712. When a pin is disposed in a set of aligned openings 720, the pin prevents the relative sliding of all of the beam elements 712 in the region of the beam elements 712 between the first end 714 of the beam elements 712 and the transverse shear constraint element 750.

FIG. 8 shows another implementation of transverse shear constraint elements 850 as a pin. The beam elements 812 shown in FIG. 8 each include channels or grooves 820 defined by the surfaces of the beam elements 812 that extend along the surface from the side of the beam element 812 (perpendicular to an axis that runs between the first end 814 and the second end 816 of the beam element 812). The channels or grooves 820 of the surface of one beam element 812 are alignable with the channels or grooves 820 of an adjacent beam element 812 to form openings defined by the two aligned channel or groove 820. The transverse shear constraint elements 850 in the compliant element 806 shown in FIG. 8 are pins that are disposable within sets of openings formed by the aligned channels or grooves 820 of the stack of beam elements 812. Thus, each pin disposed in an opening prevents the relative sliding of the beam elements 812 defining the channels or grooves 820 in the region of the beam elements 812 between the first end 814 of the beam elements 812 and the transverse shear constraint element 850. Multiple transverse shear constraint element 850 can be disposed in different openings defined by aligned adjacent channels or grooves 820 to fine-tune the flexibility of the compliant element 806.

In some implementations, the hindfoot component includes the compliant element, or both the hindfoot component and the forefoot component include the compliant element.

Some implementations include an ankle foot orthosis that includes a compliant element, similar to those disclosed above. The ankle foot orthosis includes a shank component and a foot component, and the compliant element is coupled to one of the shank component or the foot component at the mounting portion and to the other of the foot component or the shank component at the second end of at least one of the beam elements.

In a system of cantilevered and stacked beams (which are permitted to slide relative to one another), the bending stiffness of the system is less than that of a single cantilevered beam with an equivalent total thickness. Stated alternatively, a system of stacked cantilever beams, in which the beams may slide relative to one another, is more compliant than the same system of beams in which the beams are prevented from sliding relative to one another (preventing the relative sliding of the beams essentially “fuses” the beams into a single structural element). The devices, systems, and methods disclosed herein leverage this phenomenon to create a compliant member with adjustable stiffness. Specifically, the transverse shear constraint element is utilized to prevent the relative sliding of the beam elements across a region of the beam elements. As such, the region in which the beam elements cannot slide relative to one another will become stiff while the region in which the beam elements are permitted to slide will remain compliant. These two regions of the beams are in series with one another, and as such, the total beam stiffness is a function of the stiffness of these two regions of the beams. The transverse shear constraint element may be repositioned along the length of the beam elements to change the relative lengths of the stiff and compliant regions of the spring element. This concept is depicted in FIG. 1 in which the relative stiffness of two spring elements with different locations of the shear constraint element is shown. In FIG. 1A, the transverse shear constraint element is located near the second ends of the beam elements, thereby creating a long region in which the beams may not slide relative to each other and a short region in which the beams may slide relative to one another. As such, the spring component in FIG. 1A is significantly stiffer than the one depicted in FIG. 1B which has its shear constraint element located near the first end of the beam elements.

The devices, systems, and methods disclosed herein have a number of benefits relative to other mechanisms that are commonly used to vary stiffness which make it advantageous for application in a prosthetic or orthotic device. One such benefit is the manner in which stress is distributed within the spring element. When subject to load, the more compliant region of the spring element (the section of the beam distal to the transverse shear constraint element) distributes stress among the various beam elements such that no single beam is subject to all of the applied load. This advantageous distribution of stress allows for very compliant beam behavior to be achieved without exceeding the stress limits of the material. Furthermore, distributing the beam stress across multiple beam members also increases the energy storage density of the spring element, allowing the structure to store more potential energy without yielding. It should also be noted that the disclosed devices adjust the bending stiffness of the spring element and not a rotational stiffness about a single axis of rotation. This variation of bending stiffness as opposed to rotational stiffness allows the disclosed devices to more faithfully emulate the function of commercial prosthetic devices which do not have a single axis of rotation. Moreover, the stiffness adjustment mechanism of the disclosed devices and systems may be both small and lightweight, unlike many mechanisms utilized in current devices. Namely, the transverse shear constraint element may be a small structure that is able to be positioned at various positions along the length of the beam members. Unlike the propped cantilever beam mechanism utilized in some current devices, this stiffness adjustment mechanism does not require a stationary grounded structure to be present along the length of the compliant beam. Eliminating this stationary structure allows the disclosed devices and systems to be both small and lightweight relative to current devices.

In some implementations, the devices disclosed herein can be utilized as a compliant forefoot component in a prosthetic foot as shown in FIG. 2. In these implementations, the mounting portion is the segment of the prosthetic device that attaches to the shank body segment or prosthetic socket. The implementation further includes a spring component constructed from multiple stacked beam elements. In the implementation, the beam elements are of differing lengths, thicknesses, and materials to achieve the desired compliant behavior. Specifically, a compliant beam element is placed in the middle of a stack of stiffer beam elements. The beam elements in the implementation are also flat. Moreover, in the implementation, the transverse shear constraint element includes a clamping mechanism which applies a compressive load to the beam elements in a direction so as to increase the friction force between the beam elements. This transverse shear constraint element may be repositioned along the length of the beam elements by clamping this mechanism in various positions along the length of the beam elements. The prosthetic foot of the implementation is shown in a compliant configuration in FIG. 3 (transverse shear constraint element located near the mounting portion) and is shown in a stiff configuration in FIG. 4 (transverse shear constraint element located near the second end of the beam elements). In the implementation, the spring component is loaded at its free end when biomechanical loads are applied near the toe region of the prosthetic foot.

In some implementations, the transverse shear constraint element is a clamp mechanism that serves to clamp the beam elements together, thereby preventing any relative sliding between the plates. This clamp may be implemented in a variety of ways such as via a screw-based clamp (see FIGS. 2-4) or a cam-driven clamp mechanism (see FIG. 5). Other mechanisms may be utilized to drive the clamp from a variety of directions including the mechanism depicted in FIG. 6, wherein a screw is driven along an axis perpendicular to the clamping direction. Driving this screw applies a clamping force through the clamps via a wedge-based transmission mechanism. Positioning the axis of the clamp input to be different from that of the clamping direction may allow for the clamp to be more easily actuated in a clinical setting. Although the specific implementations presented to this point include clamp-based mechanisms, the transverse shear constraint element may consist of any mechanism that prevents the relative sliding of adjacent beam elements. Various examples of such mechanisms are discussed briefly herein, but the examples provided here are not an exhaustive list. One method for preventing the relative sliding of beam elements may include utilizing shear pins that can be inserted into positive engagement features of adjacent beam elements, either through the faces of the beams (see FIG. 7) or into the sides of the beams (see FIG. 8). Another approach for preventing sliding between adjacent beams is to utilize a vacuum-based system in which low pressure is generated between adjacent beams thereby allowing atmospheric pressure to compress the beams relative to one another, increasing the friction force between beams. An additional approach for preventing sliding of adjacent plates is to utilize at least one electrostatic clutch in which an electric potential is generated across two adjacent beams, thereby generating an attractive force between the two beams and increasing the friction force between the beams.

In some implementations, the beam elements are flat beams of various lengths, thicknesses, and materials. However, these beam characteristics are not restrictive. For example, the beam elements may have various combinations of shapes, lengths, thicknesses, and materials. The beam elements may be curved in the sagittal or frontal planes or have thicknesses that vary as a function of length. The beam elements may be constructed from a variety of materials including, but not limited to, carbon fiber, fiberglass, Kevlar, or nylon. The frictional properties of the materials may also be modified to achieve desired functional behavior. For example, low and/or high friction coatings may be applied to the beam elements. Low or high friction materials may also be inserted between the beam elements to modify the frictional behavior of the mechanism. Moreover, soft and low friction materials may be embedded in the beam elements such that, when a clamping pressure is applied, the high friction materials of adjacent beams are able to make contact, thereby increasing the friction force between beams. A diagram depicting this embedding of high friction material is shown in FIG. 9. Other combinations of stiff and compliant materials with various friction properties may be utilized in a similar manner such that the friction properties of adjacent plates changes with applied compressive loads. Similarly, adjacent beams may have positive engagement features such as teeth machined into their faces and a compliant spacer component may be introduced between adjacent beams. In this way, when a clamping force is applied, the compliant spacer is compressed, and the positive engagement features of adjacent beams are able to engage, preventing relative sliding of the beams.

In some implementations, the beam elements include various different materials to achieve desired properties. For example, a soft material with a low bending modulus may be placed in the middle of a stack of stiffer beams in order to increase the range of stiffness values that can be achieved by the compliant element. This process of combining a soft “core” material and stiffer outer materials into a single beam stack leverages the benefits of an area of beam mechanics known as sandwich theory. Specifically, when the compliant element is in the low stiffness configuration (transverse shear constraint element close to the first end of the beam elements), the various beams are able to slide relative to one another and the soft middle beam does not contribute significantly to the overall compliant member stiffness. However, in the stiff configuration, the soft middle beam works to position the stiff beams far from the neutral axis of the effectively “fused” beam, allowing the stiff outer beams to generate a very high stiffness relative to the case in which the soft middle beam was not present. In this way, the range of achievable stiffnesses is increased through the use of a soft middle beam in conjunction with stiff outer beam elements.

The devices, systems, and methods disclosed herein are compliant elements of a prosthesis or orthosis capable of adjusting its bending stiffness. Some non-limiting examples of applications of the devices, systems, and methods disclosed herein are in a prosthetic forefoot (FIG. 2), hindfoot, tall build height foot (FIG. 10), or low-profile prosthetic foot. Other prosthetic applications include their use in a running specific prosthetic foot or a posterior mount prosthetic device (FIG. 11). One, non-limiting application of the devices, systems, and methods disclosed herein in the area of orthotics is its application in an adjustable stiffness ankle foot orthosis in which the compliant member is configured between the foot and shank body segments (FIG. 12).

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A compliant element for an ankle and foot prosthesis or orthosis comprising:
a mounting portion;
a spring component including two or more stacked beam elements, each beam element having a first end and a second end opposite and spaced apart from the first end, wherein the first end of each of the beam elements is fixedly attached to the mounting portion; and
a transverse shear constraint element located between the first end and the second end of the beam elements, wherein the transverse shear constraint element prevents relative sliding of at least two beam elements in a region of the beam elements between the first end of the beam elements and the transverse shear constraint element.

2. The compliant element of claim 1, wherein the transverse shear constraint element is movable between the first end and the second end of the beam elements.

3. The compliant element of claim 1, wherein the transverse shear constraint element prevents the relative sliding of all of the two or more stacked beam elements in the region of the beam elements between the first end of the beam elements and the transverse shear constraint element.

4. The compliant element of claim 1, wherein the transverse shear constraint element prevents the relative sliding of a subset of the beam elements between the first end and the second end of the beam elements.

5. The compliant element of claim 4, wherein the transverse shear constraint element is configured to selectively engage various subsets of beam elements.

6. The compliant element of claim 1, wherein the transverse shear constraint element includes a clamping mechanism.

7. The compliant element of claim 6, wherein the transverse shear constraint element is a clamp that is actuated by a cam mechanism.

8. The compliant element of claim 1, wherein at least one of the beam elements comprises a different material from at least one other beam element.

9. The compliant element of claim 1, wherein the beam elements have a constant thickness between the first end and the second end of the beam elements.

10. The compliant element of claim 9, wherein the beam elements are straight.

11. The compliant element of claim 1, wherein the beam elements are curvilinear.

12. A prosthetic foot comprising the compliant element of claim 1.

13. The prosthetic foot of claim 12, wherein the prosthetic foot comprises a hindfoot component and a forefoot component, wherein the compliant element comprises at least one of the hindfoot component or the forefoot component.

14. An ankle foot orthosis comprising the compliant element of claim 1, wherein the ankle foot orthosis includes a shank component and a foot component, wherein the compliant element is coupled to one of the shank component or the foot component at the mounting portion and to the other of the foot component or the shank component at the second end of at least one of the beam elements.
